(19) United States
(12) Patent Application Publication
(10) Pub. No.: US 2005/0074780 A1
(43) Pub. Date: Apr. 7, 2005

(54) Device for analyzing slides

(76) Inventors: Marie D. Bryhan, Lindley, NY (US); Everett W. Coonan, Painted Post, NY (US); Yulong Hong, Painted Post, NY (US); Peter Knowles, Elmira, NY (US); Fang Lai, Painted Post, NY (US)

Correspondence Address: CORNING INCORPORATED, SP-TI-3-1, CORNING, NY, 14831, US

(21) Appl. No.: 10/677,950

(22) Filed: Oct. 1, 2003

(51) Int. Cl. C12Q 1/68; C12M 1/34

(52) U.S. Cl. 435/6; 435/287.2

(57) ABSTRACT

A device for analyzing slides in a microplate format is provided. The device comprises a base with slots for holding a number of planar substrates, which may be spotted with at least an array of biological or chemical molecules of interest. The base is configured to be compatible for use with high-throughput plate-readers or microplate-adapted scanners.

### FIELD OF INVENTION

The present invention relates to a device or tool useful for interrogating or processing chemical, biochemical, or biological assays. More particularly, the device enables researchers to use conventional plate-readers to interrogate material composition, surface chemistry, and biochemical reaction on the surfaces of thin planar substrates, such as glass slides, at high-throughput and can be manipulated by robotic automation for handling microplates.

### BACKGROUND

Initially, microarrays served as important tools for performing genome-wide analysis of gene expression and function. Now, microarrays come in many different forms, depending on the kinds of materials deposited or printed on the arrays (e.g., oligonucleotides, DNA, proteins, antibodies, lipid or protein membranes, receptors, cells or cell lysate, etc.). Common to all microarrays is a solid support or substrate to which the materials are attached. The substrate typically is a standard 1 inch×3 inch microscope slide. Slide-based microarray analysis is widely used in a variety of biological and chemical assays. For instance, the completion of sequencing of the human genome, as well as the genomes of many other organisms, has advanced mRNA expression profiling. Thousands of cDNA or oligonucleotides can be printed on a single glass slide and the regulations of those genes under a defined condition can be profiled simultaneously. For pharmaceutical drug discovery operations, many more drug target candidates can be identified in the coming years by means of the microarray technology.

Depending on applications, slides may be made of different materials. The characteristics of these materials often can affect the ability of some instruments to detect the biological deposits or microspots, and/or the kinds or nature of reactive surface chemistries that may be employed. To search for an ideal material with desired optical properties, it is often necessary to screen a large number of material compositions. But, assembling those materials on the bottom of microplates or making them into slides and testing them by an array scanner could be labor intensive and time consuming.

Similarly, in view of the varieties of surface chemistries that have been developed for immobilizing biological materials on a glass slide, assessments of surface chemistries can also be cumbersome. To monitor the quality of coating process, it is necessary to treat the surface and quantify the signals generated from the treatment, which often can not be done with an array scanner. For example, colloid gold staining is often used as an indirect measure of amine density of γ-amino-propyl-silane (GAPS) coated slides. A scanner can not measure the absorbency of the stained glass.

Another issue is that a number of different chemistries which have been developed on the glass slide surface for a variety of applications can not be easily adapted to polymer surfaces. Even a simple transfer of the existing surface chemistry from glass slides to glass bottom microplates is not easy or straight forward because the polymer part may interfere with the coating property. Silicone chambers from Sigma offer a medium throughput solution with which 12 virtual wells can be formed on a single slide, and up to 12 assays could be processed simultaneously. After washing and drying, the slide was scanned using a conventional slide-array scanner, which is widely available. Although this process may mimic microplate-based assays, the apparatus does not allow one to us a plate-reader to read the signal from the slide. At present, workers in the field are forced to purchase separate components from different suppliers and jury-rig the components together with a sheet of adhesive, such as available from Grace BioLabs, Inc. Such contraptions are both difficult to use, since once assembled they can not be disassembled, and suffer potential contamination from the adhesive. A more cost-efficient, simple and versatile high-throughput platform is thus desirable.

Currently, array scanners are available only with a limited choice of light sources. To develop new biological assays, many different light sources may have to be tested. Such resources are often available only with plate-readers. Also, a few imaging systems, such as the ArrayScanner™ developed by Cellomics, can only detect signals from the bottom of microplates. Hence, it can be problematic with the instrument if one wants to test different compositions or different chemistries, which have been only developed for glass slides. Tools that permit one to test feasibly of assays with a plate-reader will certainly facilitate assay development, which is often a bottle neck in the high throughput screening (HTS) labs. The present invention can address all of the aforementioned problems, and creates a device that permits easy conversion of 4 slides into a standard microplate, which can be used in any plate-reader or a microplate-adapted scanner.

### SUMMARY OF THE INVENTION

The present invention pertains in part to a platform or device for processing or interrogating biochemical, biological and chemical assays. The platform comprises a frame having a base with recesses that can accommodate a number of planar substrates. The frame is characterized as a tool for either: a) determining relative quality of said substrates qualitatively or quantitatively, b) screening of materials that have desired optical properties, or c) performing multiple, discrete assays on a slide; and said frame is designed to work in conjunction with a plate-reader or a scanner adapted for microplates. The device is divided into sections side by side. Into each section one can place a typical 1″×3″ microscope slide. The platform, preferably, has the dimensions of a standard microplate, preferably, with the size of about 5.1 inches (L)×3.25 inches (W)×0.56 inches (H). According to a particular embodiment, the slide may serve as the bottom of the plate, equivalent in area to about a quarter of a standard microplate, or about 3×8 microwells (96-well microplate format). A glass slide is elevated slightly above the bottom, where a peripheral skirt to the slide-holder supports the entire device off of, for instance, the surface of a work bench or table top, upon which the device may rest.

The device can be used as a new platform for high throughput analysis, since the device has a number of sections into which a number of planar substrate may be placed. In a preferred embodiment, the device has four (4) sections, each of which can hold a microscope slide. On the surface of a single slide, one can place a number of samples to be assayed. The configuration of the samples may be in the form of an ordered array or randomly located, depending on which instrument is to be used. In some embodiments, it is envisioned that each slide will have a possible capacity of 24 locations per slide. The overall surface area of a combined configuration of substrates creates an area equivalent to the footprint of a standard microplate. That is, the combined four slides creates a virtual microplate with a possible 96-locations. Alternate possible iterations for the four slides in combination within the slide-holder can form a virtual microplate with any industry-standard footprint, such as 96, 384, or 1536 locations, which can be handled with standard robotics commonly used in an automated laboratory. Other embodiments may have planar substrates (e.g., coated-glass, membrane on a solid support, polymers, etc.) that vary in size from a standard 1×3 inch slide to a sheet that can contain an entire virtual microplate of an industry-standard footprint. One advantage of the current design of the frame is that it accommodates dual use (with or without well blocks).

In certain embodiments, a thermoplastic material is employed to injection-mold the device. To avoid distortions or wobble resulting from stresses or contractions in the slide-holder after molding and cooling, which can detrimentally effect the desired even, planar configuration of the slides, a glass filling (e.g., frit) may be incorporated into the thermoplastic materials. The presence of glass frit in the molding materials may help to more closely match the coefficient of thermal expansion (CTE) of the plastic materials with the slides, and reduce issues associated with optical or physical distortions.

According to another aspect, the present invention pertains to methods for performing a variety of high-throughput analyses, including, substrate quality control, or genomic or proteomic assays. According to a general embodiment, the method for performing assays comprises: a) either prepare at least a set of samples on a major surface of a slide, or providing a slide already with prepared samples; b) providing a frame having a base in which a number of microscope-sized slides can fit side by side; c) placing said prepared slide into a recess in said base of said device; d) loading either biological analytes or chemical reagents onto each slide; e) performing an assay; and f) placing said frame and slides in a plate-reader.

More specifically, the method can be employed: first, to determine the quality of a reactive chemistry on a substrate; second, to determine the uniformity of a substrate material on a solid support; or third, to screen materials that have desired optical properties. Accordingly, the method comprises: a) providing a planar substrate having a surface chemistry; b) treating said substrate to make said surface chemistry detectable, and/or defining a light source; c) providing a device that comprises: a slide-holder having a base with recesses that can accommodate a number of planar substrates; d) assemblying said planar substrate in said device; and e) detecting a signal using a plate-reader.

According to another embodiment, the array-based method comprises several steps. First, either print at least an array, preferably multiple arrays, on a surface of a slide or provide a slide already with printed arrays. Second, provide a device as described above. Third, place or load the printed slide into a recess or tray in the base of the device. The slide can form the bottom surface of a virtual microplate. Fourth, contact samples of biological or chemical analytes or reagents onto of the virtual microplate. The samples can be all of the same material or each of a different material. Then, perform an assay. After the assay has run, remove the slide from the device. If appropriate, wash and dry the slides. Finally, view and analyze the assay results from the slides, such as using a standard array scanner, or place the slides back in the device and visualize the assay results with an imaging system that can directly read microplates. The same procedure may be followed for a non array-based assay. Instead of printing arrays, one can spot samples to be analyzed onto a slide in a 96/384-well format, and then analyze the assay results using a plate-reader. In other embodiments, the sequence of the first four steps may be reordered.

Since the present inventive device has the same dimensions as that of a standard microplate, one can use any robotic automation designed for handling microplates to interrogate or otherwise manipulate the device. All plate-readers and some imaging systems can also accept it. With this device, for instance, one not only can measure the coating efficiency but also can quantify the coating uniformity of slides. The device is cost-effective, easy to make and work with for end-users.

Additional features and advantages of the present invention will be explained in the following detailed description. It is understood that the foregoing and following descriptions and examples are merely representative of the invention, and are intended to provide an overview for understanding the invention as claimed.

### BRIEF DESCRIPTION OF THE DRAWINGS

FIG. 1 depicts a perspective view of an embodiment of a slide-holder device according to the present invention. The slide-holder contains four microscope slides, each possibly with an array of biospots, and each biospot at a defined location on the slide surface. The spots may number, for example, from 24 to 384 spots per slide, for a combined-total potential of 96 to 1536, respectively.

FIG. 2 shows a top overview of the base plate of a slide-holder, having a number of recesses or bays to receive microscope slides arranged side by side.

FIG. 3 depicts a partial cross-sectional view of an embodiment of the base plate along one edge of the plate and shows a recess defined by at least a sidewall with a slide accommodated within the recess.

FIG. 4 is a graph representing the distribution of total relative fluorescent signals (RFU) at 615 nm on a single, porous-substrate, coated glass slide.

FIG. 5A is a schematic representation of a substrate having a number of data spots arranged in a 3×8 formation, to serve as a location guide to FIG. 5B.

FIG. 5B is an image of a γ-amino-propylsilane-(GAPS)-coated glass slide with a colloidal gold stain.

FIG. 5C is a graph summarizing the profile of the gold stain. Areas with darker stain have higher absorbency.

### DETAILED DESCRIPTION OF THE INVENTION

### Section I—Definitions

Before describing the present invention in detail, this invention is not necessarily limited to specific compositions, reagents, process steps, or equipment, as such may vary. As used in this specification and the appended claims, the singular forms “a,” “an,” and “the” include plural referents unless the context clearly dictates otherwise. It is also to be understood that the terminology used herein is for the purpose of describing particular embodiments only, and is not intended to be limiting. All technical and scientific terms used herein have the usual meaning conventionally understood by persons skilled in the art to which this invention pertains, unless context defines otherwise.

The term “microspot” refers to a discrete or defined area, locus, or spot (e.g., ˜500 μm to ˜50 μm) on the surface of a substrate, containing a biological or chemical probe. A number of microspots may be contained with the limits of a larger area or “biospot” on the substrate surface. A biospot may contain a plurality of different probe species.

The term “probe” refers to a biological or chemical entity or molecule, which according to the nomenclature recommended by B. Phimister (Nature Genetics 1999, 21 supplement, pp. 1-60.), is immobilized to a substrate surface. Preferably, probes are arranged in a spatially addressable manner to form an array of microspots.

The term “substrate” or “substrate surface” as used herein refers to a solid or semi-solid, or porous material (e.g., micro- or nano-scale pores), which can form a stable support. The substrate surface can be selected from a variety of materials.

The term “functionalization” as used herein relates to modification of a solid substrate to provide a plurality of functional groups on the substrate surface. The phrase “functionalized surface” as used herein refers to a substrate surface that has been modified to have a plurality of functional groups present thereon. The surface may have an amine-presenting functionality (e.g., γ-amino-propylsilane (GAPS) coating), or may be coated with amine presenting polymers such as chitosan and poly(ethyleneimine).

### Section II—Description

As discussed above, use of microarray technology has become more prevalent than ever, especially since the recent completion of the human genome sequence and the genome sequences of other organisms, which enables genome-wide analyses of gene expression and function. Even though microarrays may come in many different forms (e.g., DNA, oligonuclceotides, proteins, and antibodies, etc.) depending on what is deposited on the array substrate, one thing that is common to all kinds of arrays is that a standard 1×3 microscopic slide is used as the solid support.

Currently, the processing of substrates like slides is limited with respect to the kinds of equipment used to interrogate them. At present, three general types of instruments are available for optical interrogation of substrates: 1) scanners, 2) array scanners, and 3) plate-readers. Each of these types of instruments has its merits, but for simultaneous, multi-faceted analysis of a number of different substrates, these devices have been found to be wanting.

Conventional scanners often work only with substrates in a slide-format. Recently, a few scanners have been developed, which can interrogate both slides and microplates, but these instruments are expensive (e.g., costing as much as $160,000), and are rather limited in their choice of light sources. A scanner may have only a limited number of detectors (e.g., 3-5 lasers); for example, there is no slide scanner currently has a laser emitting ultraviolet light. But, to ascertain which chemistries will work, and the proper assay conditions, certain assays will require a light source at a wavelength which can not be read or used in conventional scanners since they lack the requisite light source (e.g., lamp, laser). If, for instance, background signal can be detected by a scanner, one may check the uniformity by scanning the slides in a scanner, quantifying the signal in multiple spots at many different locations, and plotting the signals against the spot locations. This process, however, is not only time consuming, but also limited by the availability of lasers within a scanner. Moreover, scanners, unfortunately, do not have the capability to perform high throughput processing of a number of slides simultaneously.

Some imagers such as the ArrayScanner™, on the other hand, have the potential to be better instruments for interrogating microarrays under faster speeds and higher-capacity. Such an imager can enable researchers to measure a greater number of data points. Yet, current array-scanner designs have some shortcoming, since they are limited to being able to read only microplates. Hence, the desire to be able to detect from not only one slide, but over a number of slides simultaneously is still unmet.

A plate-reader, on the other hand, improves over common scanner device, since a plate-reader can detect a spectrum of dyes, fluorescence or absorbance (e.g., in the range of from ˜200 nm to ˜750 nm, preferably between ˜300 nm to 650 nm); there are many choices for plate-readers coming with a variety of light sources, capable of detecting a wide range of spectrum of signals. A plate-readers, however, that are configured to accept only microplates, can not read slides and microspots (<200 nm in diameter) in an array.

A problem associated with these instruments is how to configure one or a plurality of slides into a form that can be easily read. In the past, according to one way of solving this problem, one can tape slides to the back of a holey microplate, which fits in a plate-reader. A shortcoming of such an approach is that since the holey plate dimensions do not allow for sufficient room for taping or securing the slides, slides often detach from the holey microplate during operation. Another approach is, first, to attach a two-sided adhesive sheet that has the standard 96-well format to the back of a holey microplate, followed by attaching slides to an adhesive sheet. This method can work well, but also has disadvantages; namely the technique is 1) costly, 2) less accurate in aligning the plate to the sensor, and 3) relatively cumbersome to use. To illustrate, a single adhesive sheet can cost about $25.00 (available from Grace BioLabs). Further, because of the presence of the adhesive sheet (2 mm thick), the depth of virtual wells is 2 mm deeper, which may cause inaccurate reading by a plate-reader. Assembling of three parts—a holey plate, an adhesive sheet, and a number of slides—requires skill, patience and time, which are valued but limited factors in fast-paced, high-capacity, drug-discover labs.

The present invention provides a solution to these problems. The invention satisfies the need for a device that combines the advantages of the existent instruments while avoiding the associated shortcomings of the other approaches. The present device provides a way to configure substrate slides into a format that fits a microplate footprint, while broadening the potential range of different uses for the substrates. We envision that the present apparatus could be employed to facilitate the interrogation of substrates by converting individual substrates into a plate format, so as to take advantage of array-scanners and plate-readers.

The device is a versatile platform for positioning or holding a number of individual substrates in an automation-friendly configuration compatible with standard microplate robotics. The device promotes high-throughput interrogation of biological or chemical assays that have been performed on the substrate. Conceptually, the present device is related to an appliance which could convert a number of microscope slides into a microplate with wells, as described in U.S. Provisional Patent Application No. 60/418,101, by Hong et al., the content of which is incorporated herein by reference. Unlike the previous appliance, however, we have found that for uses such as high-throughput optical interrogation, creating wells on slides is not required, and in some instances may not be desired. For example, sometimes it may be advantageous not to have wells if the light source is incident upon the slide at an angle for optical detection. Having walls defining the microplate wells may obstruct or prevent effective detection using light beams with incidences oriented at oblique angles, or certain detection techniques (e.g., evanescent-field sensors).

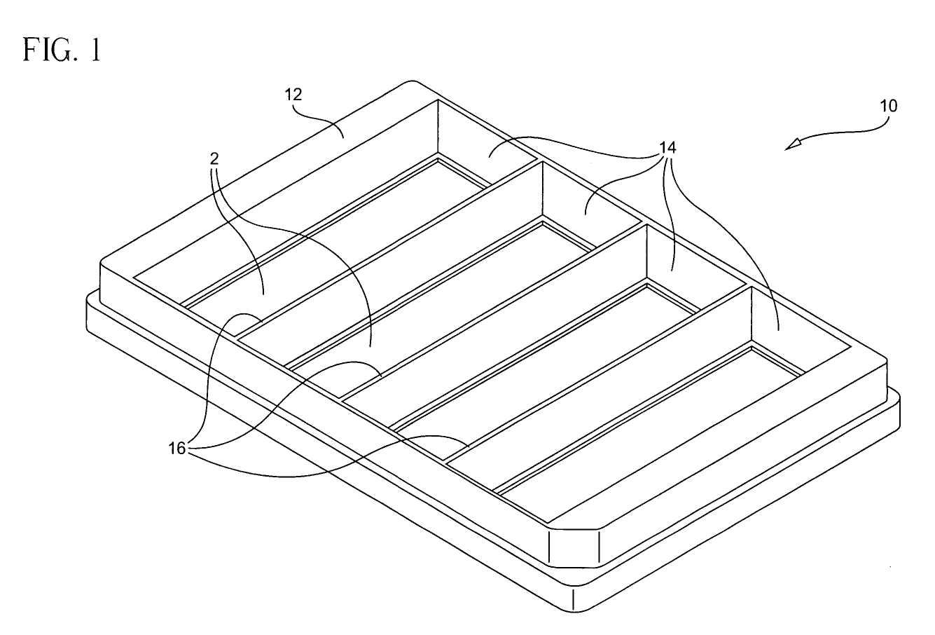

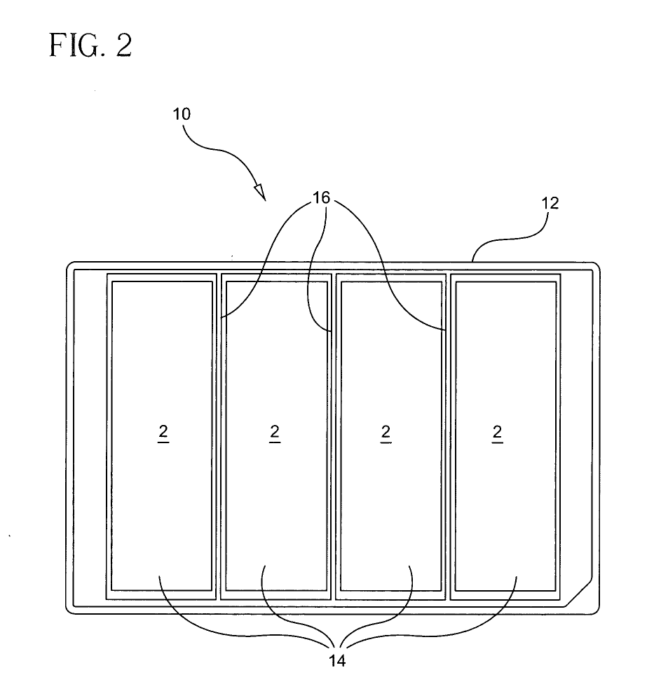

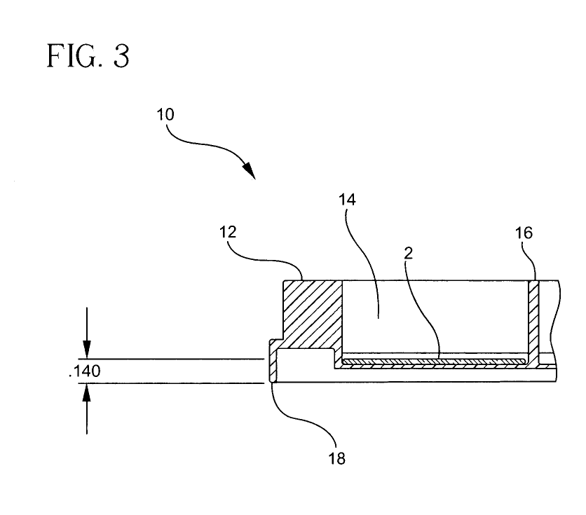

The present device is depicted in FIG. 1, which shows in perspective an overall view of an embodiment of the present device. The device 10 comprises a frame 12 or plate, which can accommodate a plurality of planar substrates 2 (e.g., slides), side by side (e.g., four (4) slides), in recesses 14 or bays. Each recess is defined by sidewalls 16. FIG. 2 represents a top overview, looking into the recesses. FIG. 3 shows a partial cross section of the device. A glass slide 2 is placed in a recess 14, parallel with the bottom plane of the frame 12. The glass surface is elevated about 0.050 to about 0.150 inches, preferably, about 0.079 to about 0.140 inches from bottom of the microplate skirt 18 to avoid contact with the work bench. If the substrate has been treated (i.e., coated or an assay has been performed on the substrate), the treated surface of the slide should be placed face-up.

It is envisioned that the frame will be machined or injection molded from a rigid, dimensionally stable plastic material, and can be designed for either reuse or one-time use. The frame can be made of a variety of materials. Examples of suitable materials may include, but not limited to: rubber, silicone, a rigid plastic (e.g. PET, polystyrene, polypropylene, olefin or polyolefin, etc.), or some other materials, preferably of a grade suitable for injection molding. In some embodiments, to avoid wobble, glass filling may be applied to thermoplastic materials used in injection-molding the device. To minimize reading signals from the device, it should also be made of the materials that have low absorbency or fluorescent signals in a wide spectrum (e.g., ˜200-700 nm). Black additive (e.g., carbon) may be used to further reduce background reading, if any.

A number of features, although not absolutely necessary, may be added to the device for easier operation. For example, a slot for each section may be introduced to one side of the device for conveniently loading and removing slides. Alternatively, a plastic “spring” for each section may be introduced to the other side for tightening a slide at its location.

Preferred embodiments, such as illustrated in FIGS. 1 and 2, may accommodate a combined-total potential of 96, 384, or 1536 array spots or sites across four slides—8×12 (96), 12×24 (384), 24×64 (1536). Other matrix arrangements or configurations are also contemplated.

The present device can be employed in a variety of particular applications. According to the invention, one use for the present slide holder can be as a quality control device. That is, the slide-holder can be used as a tool for analyzing the quantitative or qualitative parameters of the substrates' surface or reactive chemistries. For instance, porous substrates, like those described in U.S. Patent Publication Nos. 2003-0003474, or 2002-0142339, or PCT Publication No. WO 00/61282, or an article by M. Glazer et al., Colloidal Silica Films for High-Capacity DNA Probe Assays, Chem. Mater. 2001, 13, 4773-4782, the contents of each are incorporated herein by reference, can be made by applying glass frits onto a solid surface by means of either a tape-casting, screen-printing, or sol-gel method. The porous substrate can be prepared with a coating of chemical/organic molecules, etc. The uniformity of the porous substrate layer directly affects the performance of biochemical assays on the surface, especially when the solid support surface gives the signal that is to be used as a measure for an assay.

With a slide-holder like the present device, one can ascertain the quality of the porous substrate coating on the slide according to a number of techniques. One may measure fluorescent signals at a desired or predetermined wavelength to monitor the uniformity of the coating. If no detectable signal is emitted from a porous substrate, for example, one can easily determine the spectrum of the glass slide that supports the porous substrate, which will permit one to choose the proper excitation/emission wavelengths for measuring the uniformity of coating. In the case of a porous slide, a thin coating may have higher background than thick coating; therefore, the level of background can be used to measure the coating uniformity or quantity. In other words, a thicker coating will generate a greater amount of desired detection signal than a thinner coating.

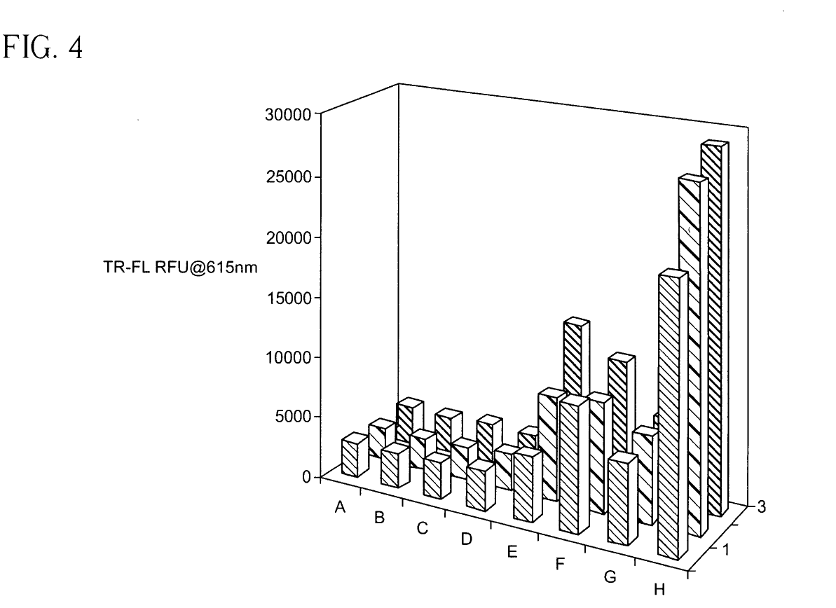

Data from an example, using time-resolved fluorescence to measure the uniformity of porous substrate, is given in FIG. 4, which depicts the distribution of total relative fluorescent signals (RFU) at 615 nm on a single porous substrate coated glass slide. Both the x-axis and the y-axis represent spot locations, and the z-axis indicates RFU. The signal is known coming from the glass slide not from the porous substrate; therefore, the area where there is strong signal is the area where coating is thinner.

For the purpose of quality control, it is often necessary to stain or treat the coated slides and quantify the stain or the treatment. When using the present invention with a plate-reader, for instance, one can obtain data signals from a plurality of locations (e.g., at least 24, preferably 32 or more locations) on a substrate, and quantify the signal distribution over the entire slide to determine uniformity of a coating. For example, a GAPS-coated glass slide can be stained by Coomassie blue, colloidal gold or some other reagents for the detection of coating efficiency and uniformity. Since either Coomassie blue or colloidal gold stains for positive charge, the stain signal is a good indirect measure of the amine density on the surface. A previous study has shown that there is a good correlation between the signal of Coomassie blue stain and the absolute amount of amines on the GAPS surface. In a study, a spectrometer (designed for using cuvette) is used to measure three different areas of one Coomassie blue stained slide to quantify the stain, which has two drawbacks. First, vast majority of the stained areas is not measured; therefore, the coating uniformity cannot be demonstrated quantitatively. Second, since it is hard to define a fixed area to be measured by adjusting the position of the clamp used to hold the slide, different areas of different slides are often measured; comparison of different slides, if not uniformly coated, is difficult.

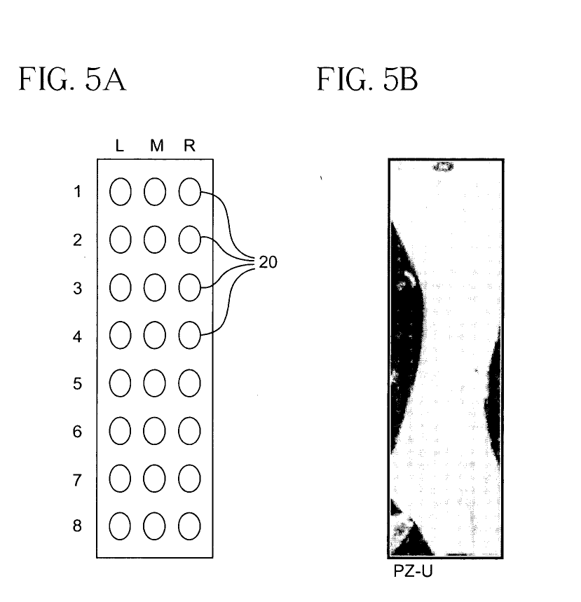

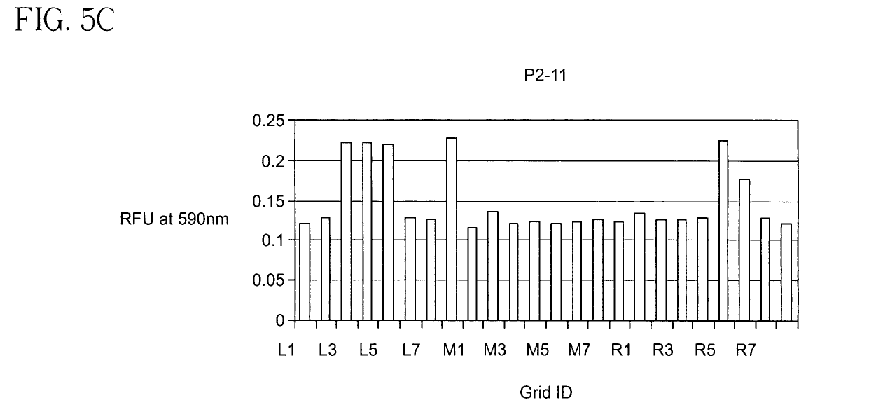

FIGS. 5A-5C demonstrate and summarize data derived from the use of the present slide holder for profiling and quantifying colloidal gold stain on a GAPS-coated glass slide (Corning Inc.). The GAPS-coated glass slide was stained with 4 ml of Protogold (BB International) at room temperature for 4 hours, rinsed in water twice, and air-dried. The stained slide was placed in the slide holder and light absorbency at 590 nm was measured using an existing method established for measuring gold stain of a Corning 96-well microplate. FIG. 5A shows a map of the slide indicating the relative location of each data point. A total of 24 data points was acquired from a single slide. The 24 data points from each slide allow one to profile the uniformity of the surface, which is a way to monitor the quality of surface coating. The average signal from the 24 data points also permits the calculation of coefficient of variations (CV), which is a good measure of variations within or between slides. FIG. 5B is an image of a gold stained GAPS-coated slide. FIG. 5C provides a graph summarizing the profile of the gold stain corresponding to each data point. The x-axis refers to the respective data points, while the y-axis presents the level of absorbency for each point. The areas with darker staining exhibited a higher level of absorbency.

In another application, the present apparatus could be a useful screening tool. Depending on applications, array slides may be made of different materials and coated with different chemistries. One can screen for materials that have desired optical properties, (e.g., level of transparency or low background signal, etc.). That is, substrates of different materials can be made into slides and placed in the holder apparatus, and one can compare the different materials in parallel. For example, to search for a glass composition that has a desired transparency, one can make 1×3 inch slides out of various compositions, test their transparency by placing them in a slide holder, and measure the absorbance of the material at a proper wavelength with a plate-reader. Similarly, one can make various materials (organic or inorganic) into a standard 1×3 inch slide, and quickly test the materials using the apparatus to discern which composition has the lowest fluorescent signal at a given wavelength. If a material proves to be useful as the bottom of microplates, one can save substantial amount of money and effort in making a microplate prototype before running actual, empirical tests.

Since microarrays are made predominately on glass substrates, most surface chemistries have been adapted or designed for a glass support surface. Microplates typically are made from other materials such as polymers, which do not have a proper surface chemistry that either facilitates or is compatible with a desired assay. Hence, a proper support to perform certain assay chemistries within a microplate can be difficult. With present device, however, one can readily test the surface chemistry. One may examine combinations of materials and their properties in a single or reduced number of screening processes. That is one can prepare a few substrates and run them together, simultaneously within the frame and compare their results.

Also, a 3×8 matrix of samples either of the same or different material can be used. One may perform assays on glass-substrates either within the frame or before placing the substrates into the frame, and detect the assay results using a plate-reader. Instead of having to manufacture beforehand an entire glass bottomed microplate, the present invention makes use of exiting components, which can provide quick and cost efficient assessment of many assay conditions.

Using the present apparatus, one can also perform, in high throughput, multiple biological assays on a single slide, either as a solidly coated surface or a porous substrate. Because small pores and channels in a porous substrate creates a capillary effect, normally one can not form virtual wells on the top of porous substrates using an insert having a number of cells or holes containing a 3×8 configuration of bottom-less wells, such as Hong et al described previously. Assay solution would pass through the pores, migrate into adjacent wells, and contaminate adjacent samples. Cross-talk can be eliminated using the present apparatus. The well-less slide holder can be used to quantify assay signals in a plate-reader, so long as the samples to be assayed are spotted in an array format (e.g., 96-spots). That is, for example, one can score or divide the porous substrate into 3×8 sections with a razorblade, and isolate those sections from each other with a resin or wax pen. If samples are carefully loaded in the center of each section, they should be in the location equivalent to the center of each well in a 96-well microplate.

Any type of single spot or array-based assays, such as for target validation, including, for example, pharmaceutical drug screening, clinical diagnostics, genomics, and proteomics may be interrogated with the device. Assays may involve the use of DNA/oligonucleotide “theme” arrays, antibody/protein/peptide “theme” array, tissue/cell array, and other small molecule arrays. As used herein the term “theme array” refers to an array having a select sample of particular biological or chemical materials of interest. The apparatus may also be used for assays with one biological or chemical molecules per area, if desired.

In addition to the foregoing applications, the present apparatus can also be used as a tool to facilitate detection of multiple arrays on a single substrate, analogous to arrays on the bottom of multi-well plates. That is, a number of microspots is arranged within the boundaries of a larger biospot on the surface of the substrate, and a plurality of the areas is located over the entire substrate. For example, cell-based assays in an array format can be helped with the invention. DNA of gene expression clones may be printed into arrays. After treating the arrays with transfection reagent and overlaying cells, cells will uptake the DNA and express the proteins encoded by the DNA at discrete locations. The intracellular location or the function of the expressed proteins may be viewed in an imager like the ArrayScanner™.

The new platform offers a number of advantages over previous systems and devices. The advantages, just to name a few, include the following. First, this device enables one to take advantage of all existing surface chemistries which may be built onto the glass slide surface. No further development or additional modification of the surface chemistry is required in order to transfer slide-based assays to microplate-based assays. Second, the new apparatus can achieve a true industry-standard microplate footprint with respect to the number of arrays or individual assays one may be able to execute by means of true parallel processing. Hence, third, the device is fully compatible with a range of standard slide or microplate-associated instruments. All of the conventional lab instruments (e.g., glass slide arrayer/scanner, microplate liquid handler, etc.) that one would likely find in an industrial, clinical or research laboratory are readily usable. Users of the inventive platform need not incur extra costs for new equipment since the new platform combines the slide-based assay and microplate-based high-throughput technologies. This feature is one of the significant advantages over other commercially available microplate-based array systems, which typically require the user to buy expensive new instrument(s). Fourth, the device has a modular design, which workers can assemble and disassemble with ease for flexibility-of-use as they may desire. Fifth, the inventive device is inexpensive and disposable.

The present invention has been described generally and in detail by way of examples and figures. Persons skilled in the art, however, will understand that the invention is not limited necessarily to the embodiments specifically disclosed, but that modifications and variations can be made without departing from the spirit of the invention. Therefore, unless changes otherwise depart of the scope of the invention as defined by the following claims, they should be construed as being included herein.

1. A device comprising:
a frame having a base having a number of recesses in which a number of planar substrates can fit side by side; said frame is characterized as a tool for interrogation of individual substrates in a plate format.

2. The device according to claim 1, wherein each of said recesses is defined by a sidewall.

3. The device according to claim 1, wherein said slide-holder can accommodate four (4) microscope slides.

4. The device according to claim 3, wherein said four slides in combination forms a virtual microplate with an industry-standard footprint.

5. A platform for performing high-throughput interrogation of biological, biochemical, or chemical assays, the device comprising: a frame having a base with recesses that can accommodate a number of planar substrates; said frame is characterized as a tool for either: a) performing multiple, discrete assays on an individual substrate, b) determining relative quality of said substrates, c) screening of materials that have optical properties; and said frame is designed to work in conjunction with a plate-reader or a scanner adapted for microplates.

6. The device according to claim 5, wherein said slide-holder can accommodate four (4) microscope slides.

7. The device according to claim 5, wherein a surface property of each substrate can be configured in an 8×12 matrix according to a 96-well foot-print.

8. The device according claim 5, wherein a surface property of each substrate can be configured in a 16×24 matrix according to a 384-well foot-print.

9. The device according to claim 5, wherein a surface property of each substrate can be configured in a 12×36 matrix according to a 1536-well foot-print.

10. A method performing assays, the method comprising:
a) either prepare at least a set of samples on a major surface of a slide, or providing a slide already with prepared samples;
b) providing a frame having a base in which a number of microscope-sized slides can fit side by side;
c) placing said prepared slide into a recess in said base of said device;
d) loading either biological analytes or chemical reagents onto each slide;
e) performing an assay;
f) placing said frame and slides in a plate-reader.

11. The method according to claim 10, wherein said samples can be all of the same material or each of a different material.

12. The method according to claim 10, further comprises viewing and analyzing the results of said assay.

13. The method according to claim 10, wherein in each slide has sections arranged in a 3×8 matrix.

14. The method according to claim 10, wherein in each slide has sections arranged in a 6×16 matrix.

15. The method according to claim 10, wherein in each slide has sections arranged in a 12×36 matrix.

16. The method according to claim 10, wherein said frame said frame is characterized as a tool for either: a) determining the quality of surface chemistry on a substrate, b) determining the uniformity of said surface chemistry, or c) screening of materials that have optical properties; and said frame is designed to work in conjunction with a plate-reader.

17. A method of determining the quality of a reactive chemistry on a substrate, the method comprising:
a) providing a planar substrate having a surface chemistry;
b) treating said substrate to make said surface chemistry detectable
c) providing a device that comprises: a slide-holder having a base with recesses that can accommodate a number of planar substrates;
d) assemblying said planar substrate in said device;
e) detecting a signal using a plate-reader.

18. The method according to claim 17, wherein said samples are all of the same material or each of a different material.

19. The method according to claim 17, further comprises analyzing the results of said assay.

20. A method of determining the uniformity of a substrate material on a solid support, the method comprising:
a) providing a planar substrate having a chemical, porous or membrane surface;
b) treating said substrate to make said surface detectable, or defining a light source;
c) providing a device that comprises: a slide-holder having a base with recesses that can accommodate a number of planar substrates;
d) assemblying said planar substrate in said device;
e) detecting a signal using a plate-reader.

21. The method according to claim 20, wherein said samples are all of the same material or each of a different material.

22. The method according to claim 20, further comprises analyzing the results of said assay.

23. A method of screening materials that have optical properties, the method comprising:
a) providing a planar substrate;
b) treating, if necessary, said substrate to make said substrate detectable;
c) providing a device that comprises: a slide-holder having a base with recesses that accommodate a number of planar substrates;
d) assemblying said planar substrate in said device;
e) detecting a signal using a plate-reader.

24. The method according to claim 23, wherein said method includes defining a light source.
